(19) United States
(12) Patent Application Publication
(10) Pub. No.: US 2026/0331212 A1
(43) Pub. Date: Oct. 22, 2026

(54) PROTEIN DESIGN USING DIFFUSION MODELS OPERATING ON FULL ATOM REPRESENTATIONS

(71) Applicant: GDM Holding LLC, Mountain View, CA (US)

(72) Inventors: Russell James Bates, London (GB); Robert David Fergus, New York, NY (US); Vinicius Zambaldi, London (GB); David La, London (GB); David William Saxton, London (GB); Zachary Wu, Milpitas, CA (US); Thomas Frerix, London (GB); Fabian Bernd Fuchs, London (GB); Rosalia Galiazzi Schneider, London (GB); Jonas Anders Adler, London (GB); Alexander E. Chu, London (GB); Simon Kohl, London (GB)

(21) Appl. No.: 19/486,752

(22) PCT Filed: May 21, 2024
(86) PCT No.: PCT/EP2024/063990

§ 371 Date: Nov. 21, 2025

Related U.S. Application Data
(60) Provisional application No. 63/468,492, filed on May 23, 2023.

(51) Int. Cl. G16B 15/00; G16B 40/00

(52) U.S. Cl. CPC G16B 15/00; G16B 40/00

(57) ABSTRACT

Methods, systems, and apparatus, including computer programs encoded on a computer storage medium, for designing proteins. In one aspect, a method comprises: generating noisy molecular structure data sampled from a noise distribution that defines, for each position in an amino acid sequence of the protein, a corresponding initial spatial position for each atom in a predefined set of possible atoms; and processing the noisy molecular structure data using a diffusion model that comprises a denoising neural network to generate denoised molecular structure data that defines a denoised version of the noisy molecular structure data.

### BACKGROUND

This specification relates to designing proteins.

A protein includes a sequence of amino acids. An amino acid is an organic compound which includes an amino functional group and a carboxyl functional group, as well as a side-chain (i.e., group of atoms) that is specific to the amino acid.

Protein folding refers to a physical process by which a sequence of amino acids folds into a three-dimensional configuration. The structure of a protein defines the three-dimensional (3D) configuration of the atoms in the amino acid sequence of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification generally describes a system implemented as computer programs on one or more computers in one or more locations that performs protein design.

The system described in this specification can design novel proteins, e.g., proteins that bind to a specified ligand, e.g., a small molecule ligand or a protein ligand. The system can include a diffusion model that, at each denoising iteration in a sequence of denoising iterations, processes a “noisy” version of a protein using a denoising neural network to predict a denoised version of the protein.

Designing a protein includes determining the amino acid sequence of the protein, i.e., determining a respective amino acid for each position in the amino acid sequence of the protein. Each amino acid is made up of atoms in a respective configuration. The system is capable of switching between or smoothly interpolating between the sets of atoms that are in play, as the identities of the amino acids included in the amino acid sequence change during the diffusion process (thus causing the atoms that make up the amino acids at each position to change).

Particular embodiments of the subject matter described in this specification can be implemented so as to realize one or more of the following advantages.

The protein design system described in this specification can implement a diffusion model that is capable of controlling all possible atoms for each possible protein residue. The system can then infer the amino acid sequence of the protein directly from the designed coordinates. The system can thus simultaneously sample novel structures and corresponding amino acid sequences. Rather than having to phase unused atoms “in and out of existence”, the system trains the diffusion model to “hide” unused atoms at a throw-away spatial position (e.g., the alpha-carbon position) of the corresponding residue, i.e., a spatial position that signifies that the atom is not part of the designed protein. The system can then automatically discard the unused atoms at throw-away spatial positions.

The protein design system can condition the de-noising process on structural information from target molecules. Conditioning is the process of guiding the diffusion model with additional context, in this case, e.g., the atomic coordinates of the target molecule. The protein design system can thus go beyond re-scaffolding, e.g., to design de novo functional protein components that act as binders or catalysts (enzymes) for target molecules.

The protein design system can jointly determine: (i) the structure, and (ii) the amino acid sequence of a protein, e.g., that is predicted to bind to a target molecule. In contrast, conventional systems generally treat these tasks separately, e.g., one conventional system may be configured to process an amino acid sequence of a protein to determine the protein structure, while another conventional system may be configured to process a protein structure to determine an amino acid sequence of a protein that realizes the protein structure. By jointly predicting the structure and the amino acid sequence of a protein in a single forward pass, e.g., using a diffusion generative machine learning model, the system can enable reduced consumption of computational resources, e.g., memory and computing power, compared to previous systems that perform these tasks separately.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example protein design system.

FIG. 2 is a flow diagram of an example process for designing a protein using a diffusion model.

FIG. 3 is a block diagram of an example diffusion model.

FIG. 4 illustrates an example embedding scheme that specifies the positions of atoms within amino acids for a protein.

FIG. 5 illustrates an example embedding scheme that specifies the positions of heavy atoms within amino acids for a protein.

FIG. 6 illustrates an example embedding scheme that specifies the positions of groups of atoms within amino acids for a protein.

FIG. 7 is a flow diagram of an example process for generating molecular structure data for a protein using a denoising neural network.

FIG. 8 is a flow diagram of an example process for training a denoising neural network to generate molecular structure data for proteins.

FIG. 9 is a flow diagram of an example process for processing denoised molecular structure data to identify atoms included within amino acids for a protein.

FIG. 10 illustrates example throw-away spatial positions for an amino acid.

FIG. 11 illustrates an example protein designed to bind with a target protein.

FIG. 12 illustrates an example protein designed to include a specified sequence of amino acids.

Like reference numbers and designations in the various drawings indicate like elements.

### DETAILED DESCRIPTION

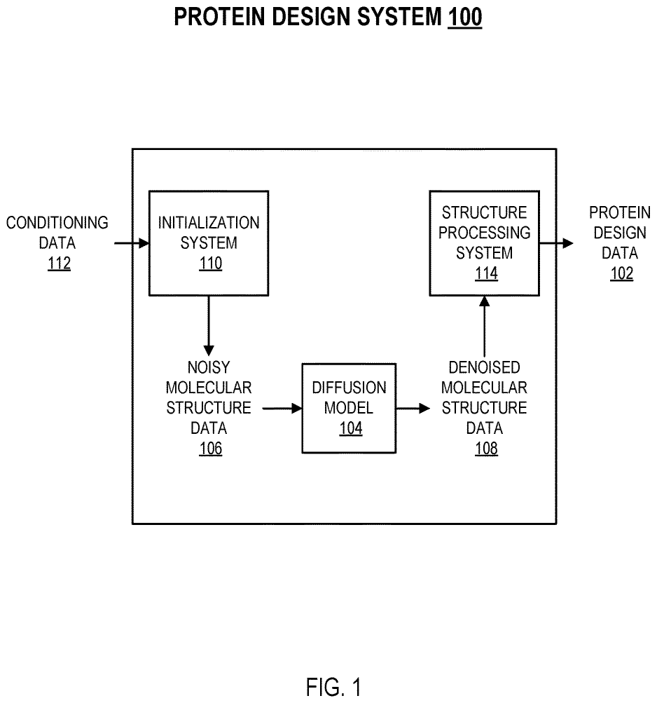

FIG. 1 shows an example protein design system 100. The protein system 100 is an example of a system implemented as computer programs on one or more computers in one or more locations in which the systems, components, and techniques described below are implemented.

The protein design system 100 can generate protein design data 102 characterizing a designed protein using a diffusion model 104. As an example, the protein design data 102 can specify a structure of the designed protein. As another example, the protein design data 102 can specify an amino acid sequence for the designed protein. As another example, the protein design data 102 can specify both: (i) the structure of the designed protein, and (ii) the amino acid sequence of the designed protein.

The system 100 can use the diffusion model 104 to design the protein for a particular function. For example, the system 100 can design the protein to bind with a specified ligand (e.g., a small molecule ligand or a protein ligand). As a further example, the system 100 can receive data characterizing the specified ligand and can generate the protein design data 102 for the designed protein based on the received data characterizing the specified ligand. As another example, the system 100 can design the protein to bind with a specified target protein.

To design the protein, system 100 can process noisy molecular structure data 106 using the diffusion model 104 to generate denoised molecular structure data 108 that characterizes the designed protein. The noisy molecular structure data 106 and the denoised molecular structure data 108 can include, for each amino acid within the amino acid sequence for the designed protein, a corresponding amino acid embedding. Each amino acid embedding can include a sequence of atom embeddings for each of a predefined set (i.e., collection) of atoms that may be included within the corresponding amino acid of the protein. The pre-defined set of atoms can comprise multiple instances of the same type of atom (i.e., element). Each atom embedding can characterize a spatial position of a corresponding atom.

The noisy molecular structure data 106 specifies initial spatial positions for each of the atoms that may be included within the amino acids of the designed protein. The diffusion model 104 processes the noisy molecular structure data 106 following a denoising process to produce the denoised molecular structure data 108 characterizing the designed protein. In some implementations, the denoising process is an iterative process, and the diffusion model 104 can produce the denoised molecular structure data 108 by iteratively denoising the noisy molecular structure data 106 over a sequence of denoising iterations. Following the denoising process, the diffusion model 104 can determine, for each amino acid of the designed protein, both (i) the positions of the atoms that may be included within amino acid and (ii) which atoms to include within the amino acid.

A particular amino acid within the designed protein may not include every atom from the predefined set of atoms that may be included within the amino acid. The diffusion model 104 can determine which atoms to include within the amino acids of the designed protein and can generate the denoised molecular structure data 108 to encode which atoms are included within each amino acid the designed protein. In particular, the diffusion model 104 can encode which atoms are included within the designed protein using the generated spatial positions of the atoms. For example, each amino acid of the designed protein can be associated with “throw-away” locations (spatial positions) for the amino acid, and the diffusion model 104 can encode that a given atom is to be excluded from the designed protein by placing the atom at a throw-away location of the amino acid.

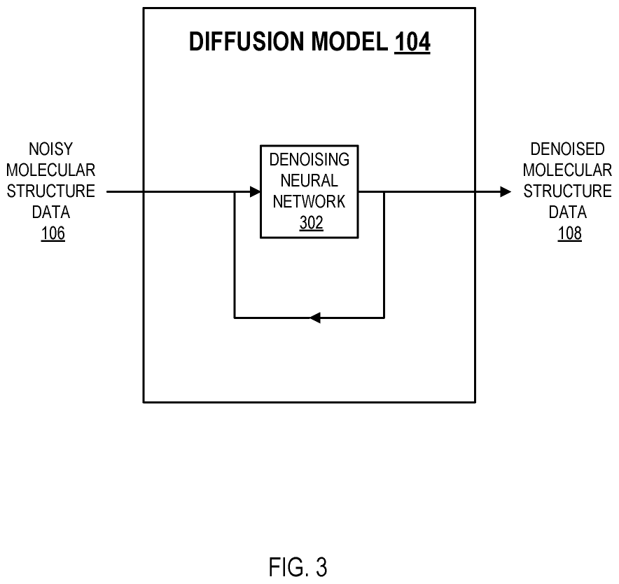

The diffusion model is described in more detail below with reference to FIG. 3.

The protein design system 100 can include an initialization system 110. The initialization system 110 can generate the noisy molecular structure data 106. In particular, the initialization system 110 can initialize the noisy molecular structure data 106 by sampling some or all of the initial spatial positions for the atoms that may be included within the designed protein from a noise distribution (e.g., a Gaussian distribution, a uniform distribution, etc.).

The initialization system 110 can receive conditioning data 112 and can generate the noisy molecular structure data 106 based the conditioning data 112.

As an example, the conditioning data 112 can specify predefined spatial positions for one or more atoms of the designed protein and the initialization system 110 can generate the noisy molecular structure data 106 to designate the atoms as being static atoms with spatial positions that are not modified by the diffusion model 104. This enables the protein design system 100 to design the protein to include the static atoms. As an example, the static atoms can be part of specified protein residues (e.g., atoms of the protein associated with a binding site of the protein) and the system 100 can design a protein that includes the specified protein residues. An example of designing a protein to include a target sequence of amino acids is illustrated below with reference to FIG. 12.

As another example, the conditioning data 112 can characterize a target molecule (e.g., a target small molecule ligand, a target protein ligand, a target protein, etc., for the designed protein) and the initialization system 110 can generate the noisy molecular structure data 106 to define a structure of the target molecule. In general, the noisy molecular structure data 106 can specify initial spatial positions for each atom of the target molecule. In particular, the noisy molecular data 106 can include one or more embeddings that characterize spatial positions of atoms of the target molecule. As another example, the conditioning data 112 can characterize an electrostatic potential or surface charge distribution that the designed protein should have or create.

When the noisy molecular data 106 includes embeddings for the target molecule, embeddings for the target molecule can share a same dimensionality as the amino acid embeddings for the designed protein. As an example, embeddings for the target molecule can be atom embeddings from the predefined set of atoms that may be included in the designed protein.

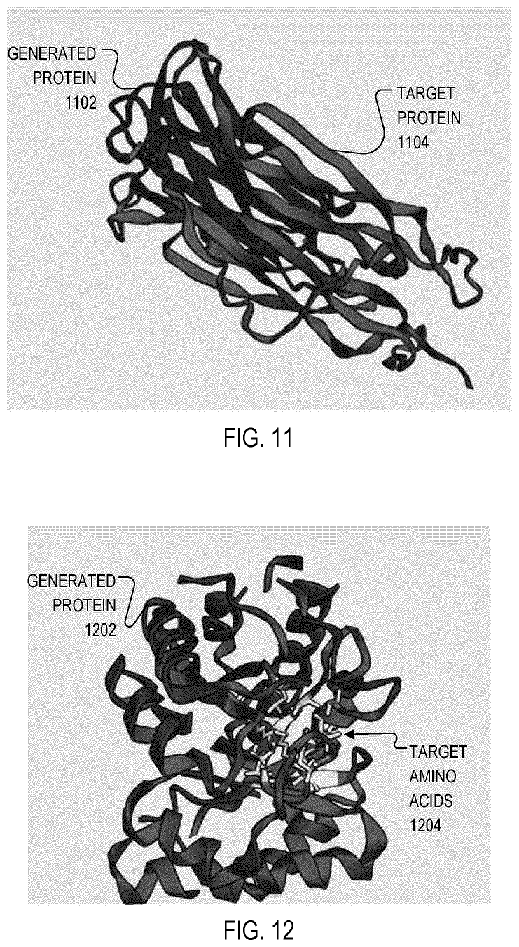

When the initialization system 110 generates the noisy molecular structure data 106 to define the structure of the target molecule, the initialization system 110 can also generate the noisy molecular structure data 106 to designate one or more atoms in the target molecule as being static atoms having predefined spatial positions that are not modified by the diffusion model 104. For example, the initialization system 110 can generate the noisy molecular structure data 106 to designate all of the atoms of the target molecule as being static atoms with predefined spatial positions that are not modified by the diffusion model 104. This enables the protein design system 100 to design the protein to bind with the target molecule. An example of designing a protein to bind with a target molecule is illustrated below with reference to FIG. 11.

The protein design system 100 includes a structure processing system 114. The structure processing system 114 can process the denoised molecular structure data 108 generated by the diffusion model 104 to produce the protein design data 102.

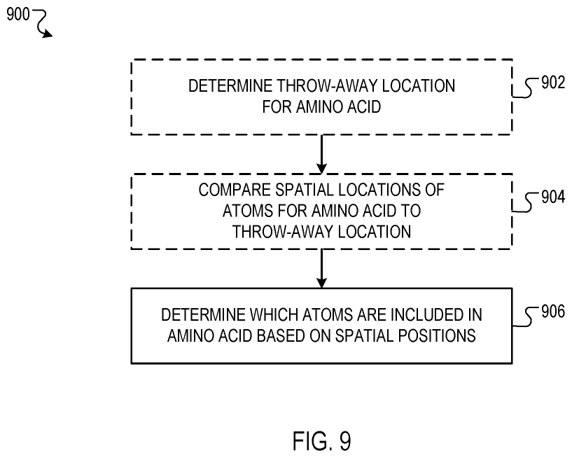

As described above, the denoised molecular structure data 108 can encode which atoms are included within each amino acid the designed protein. The structure processing system 114 can process the denoised molecular structure data 108 to remove atoms that are excluded from the designed protein. For example, when the denoised molecular structure data 108 encode which atoms are included within the designed protein using the generated spatial positions of the atoms, the structure processing system 114 can remove atoms based on their generated spatial positions. An example process for removing atoms based on their generated spatial positions is described in more detail below with reference to FIG. 9.

The structure processing system 114 can perform a variety of operations process the denoised molecular structure data 108 to determine the identities of the amino acids in the amino acid sequence of the designed protein.

For example, the structure processing system 114 can use a predefined mapping between sets of atoms and amino acids to determine the identities of the amino acids in the amino acid sequence of the designed protein. As a further example, when the structure processing system 114 excludes atoms from the designed protein, the structure processing system 114 can determine an identity of each amino acid within the designed protein based on the remaining atoms for the amino acid using the predefined mapping.

As another example, the structure processing system 114 can include a sequencing machine learning model and can process the denoised molecular structure data 108 with the sequencing machine learning model to determine an amino acid sequence. For example, the diffusion model 104 may erroneously exclude or include atoms within the denoised molecular structure data 108 and the structure processing system 114 can use the sequencing machine learning model to determine a corrected amino acid sequence for the designed protein. As another example, the diffusion model 104 may generate an approximate spatial structure for the designed protein, and the structure processing system 114 can use the sequencing machine learning model to generate a final amino acid sequence for the designed protein based on the approximate spatial structure generated by the diffusion model 104. An example sequencing machine learning model is described by Dauparas et al. in “Robust Deep Learning-Based Protein Sequence Design Using ProteinMPNN”, Science 6615 (378), p 49-56 (2022).

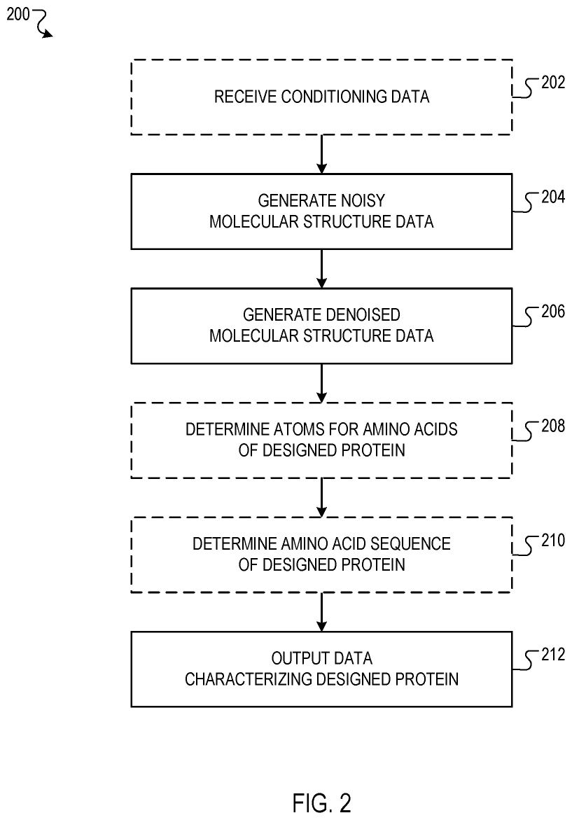

An example process for designing a protein using the diffusion model 104 is described in more detail below with reference to FIG. 2.

After designing the protein, the protein design system 100 can output the protein design data 102 to an external system in order to synthesize the designed protein. For example, the protein design data 102 can specify the amino acid sequence of the designed protein, and the external system can synthesize a protein having the specified amino acid sequence. As a further example, the protein design data 102 can include instructions for synthesizing the designed protein (e.g., required conditions for synthesizing the designed protein) and the external system can synthesize the designed protein as instructed.

FIG. 2 is a flow diagram of an example process for designing a protein using a diffusion model. For convenience, the process 200 will be described as being performed by a system of one or more computers located in one or more locations. For example, a protein design system, e.g., the protein design system 100 of FIG. 1, appropriately programmed in accordance with this specification, can perform the process 200.

In some implementations, the system can receive (e.g., from a user) conditioning data for designing the protein (step 202). The system can generate the designed protein based on the conditioning data. As an example, the conditioning data can specify predefined spatial positions for one or more atoms (e.g., atoms of specified protein residues) of the designed protein and the system can design the protein to include the specified atoms. As another example, the conditioning data can characterize a target molecule (e.g., a target small molecule ligand, a target protein ligand, a target protein, etc., for the designed protein) and the system can design the protein to bind with the target molecule.

The system can generate noisy molecular structure data that defines initial spatial positions for atoms that may be included in the designed protein (step 204). In particular, for each amino acid within the designed protein, the noisy molecular structure data can specify spatial positions for each of a predefined set of atoms that may be included within the amino acid.

The predefined set of atoms that may be included within the amino acid can include each atom present (e.g., backbone atoms, sidechain atoms, etc.) within a predefined set of amino acids (or amino acid residues). That is, each of the atoms in the predefined set of atoms may correspond to a respective one of the atoms of at least one of the amino acids in the predefined set of amino acids. Thus, the predefined set of atoms can comprise multiple instances of the same type of atom (i.e. element). For example, if the predefined set of amino acids were to consist of glycine and alanine residues, then the predefined set of atoms may include 3 carbon atoms, 5 hydrogen atoms, 1 nitrogen atom and 1 oxygen atom, such that the predefined set of atoms can be used to represent either a glycine or an alanine residue. As a particular example, the predefined set of atoms can be more than 30 atoms (e.g., 37 atoms, 39 atoms, 41 atoms, etc.) present within the following set of amino acids: alanine, cysteine, aspartic acid, glutamic acid, phenylalanine, glycine, histidine, isoleucine, lysine, leucine, methionine, asparagine, pyrrolysine, proline, glutamine, arginine, serine, threonine, selenocysteine, valine, tryptophan, and tyrosine.

In general, the predefined set of atoms can include more atoms than are present in any individual amino acid of the protein. After denoising the noisy molecular structure data, the system can determine which atoms are excluded from each amino acid of the protein and can determine an identity of the amino acid based on the proper subset of the predefined set of atoms that are included within the amino acid. An example process for determining which atoms are excluded from amino acids of the protein is described in more detail below with reference to FIG. 9.

The noisy molecular structure data can designate one or more atoms as being static atoms having predefined spatial positions. For example, when the system receives conditioning data that specifies predefined spatial positions for one or more atoms of the designed protein, the noisy molecular data can include the specified predefined spatial positions and designate the corresponding atoms as being static atoms of the protein. As another example, when the system receives conditioning data that characterizes a target molecule (e.g., a target small molecule ligand, a target protein ligand, a target protein, etc., for the designed protein), the noisy molecular data can characterize specified predefined spatial positions for atoms of the target molecular and designate the corresponding atoms of the molecule as being static atoms.

The noisy molecular structure can include an amino acid embedding for each amino acid within the designed protein. Each amino acid embedding can include a respective atom embedding for each of the set of predefined atoms. Each atom embedding can be a set of numerical values that characterizes a spatial position of the corresponding atom.

When the noisy molecular structure data characterizes positions of atoms for a target molecule of the designed protein, the noisy molecular structure data can include atom embeddings for each atom of the target molecule. The atom embeddings for the atoms of the target molecule can share a same dimensionality as the atom embeddings for atoms of the designed protein. In particular, the noisy molecular structure data can characterize the target molecule using the set of predefined atoms and can use a common atom embedding scheme to represent both the atoms of the target molecule and the atoms of the designed protein.

In some implementations, the system can generate the noisy molecular structure data by sampling initial spatial positions for some or all of the atoms of the designed protein from a noise distribution for the atom embeddings (e.g., a Gaussian distribution, a uniform distribution, etc.). In particular, the system can sample values for the atom embeddings within the noisy molecular structure data. When the noisy molecular structure data designates certain atoms as being static atoms with predefined spatial positions, the system can system can use the predefined spatial positions for the static atoms rather than sampling initial spatial positions for the static atoms.

The system can process the noisy molecular structure data using a diffusion model to generate denoised molecular structure data (step 206). The denoised molecular structure data defines a denoised version of the noisy molecular structure data and can specify final positions of atoms within the designed protein. An example diffusion model is described in more detail below with reference to FIG. 3.

In some implementations, when the noisy molecular structure data designates certain atoms as being static atoms with predefined spatial positions, the system can hold the spatial positions of the static atoms fixed when generating the denoised molecular structure data. In particular, the system can ensure that the denoised molecular structure data specifies the static atoms as having the same predefined spatial positions as specified by the noisy molecular structure data.

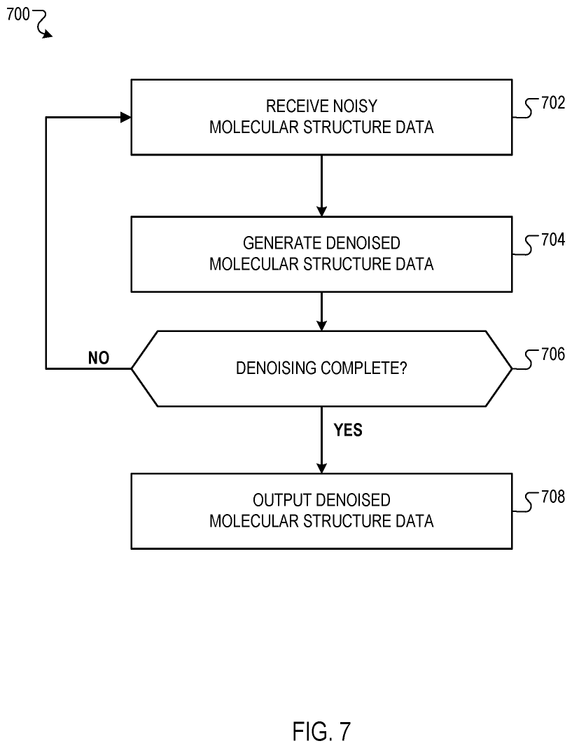

In some implementations, the diffusion model can include a denoising neural network and the system can iteratively denoise the noisy molecular structure data over a sequence of denoising iterations using a denoising neural network to generate the denoised molecular structure data. An example process of iteratively denoising noisy molecular structure data using the denoising neural network is described below with reference to FIG. 7.

In some implementations, the system can process the denoised molecular structure data to determine atoms that are included within the designed protein (step 208). In particular, for each amino acid of the designed protein, the system can identify a proper subset of the predefined set of atoms as being included in the amino acid based on the denoised molecular structure data. An example process for processing the denoised molecular structure data to identify atoms included within amino acids for the designed protein is described in more detail below with reference to FIG. 9.

In some implementations, the system can process the denoised molecular structure data to determine an amino acid sequence of the designed protein (step 210). In general, the system can process the denoised molecular structure data to determine a respective identity for each amino acid within the amino acid sequence of the protein. As an example, the system can process some or all of the denoised molecular structure using a sequencing machine learning model to generate data defining the respective identity for each amino acid in the amino acid sequence of the protein.

As another example, the system can store a mapping from proper subsets of the set of predefined atoms to amino acid identities for a predefined set of amino acids. When the system identifies, for each amino acid of the designed protein, a proper subset of the predefined set of atoms as being included in the amino acid, the system can determine the identity of the amino acid using the stored mapping. As an example, when an amino acid of the designed protein includes a same set of atoms as a particular amino acid from the predefined set of amino acids, the system can identify the amino acid of the designed protein as being the particular amino acid from the predefined set of amino acids. As a further example, for each amino acid of the designed protein, the system can identify the amino acid as being a particular amino acid from the predefined set of amino acids that maximizes a measure of similarity (e.g., a Jaccard index) between the set of atoms included within the amino acid of the designed protein and the set of atoms included within the particular amino acid.

The system can finally output data characterizing the designed protein (step 212). In particular, the system output data characterizing the designed protein (e.g., the amino acid sequence of the designed protein, the structure of the designed protein, synthesis instructions for the designed protein, etc.) to a protein synthesis system, and the protein synthesis system can synthesize a protein having the amino acid sequence of the designed protein.

FIG. 3 is a block diagram of an example diffusion model 104. The diffusion model 104 is an example of a system implemented as computer programs on one or more computers in one or more locations in which the systems, components, and techniques described below are implemented.

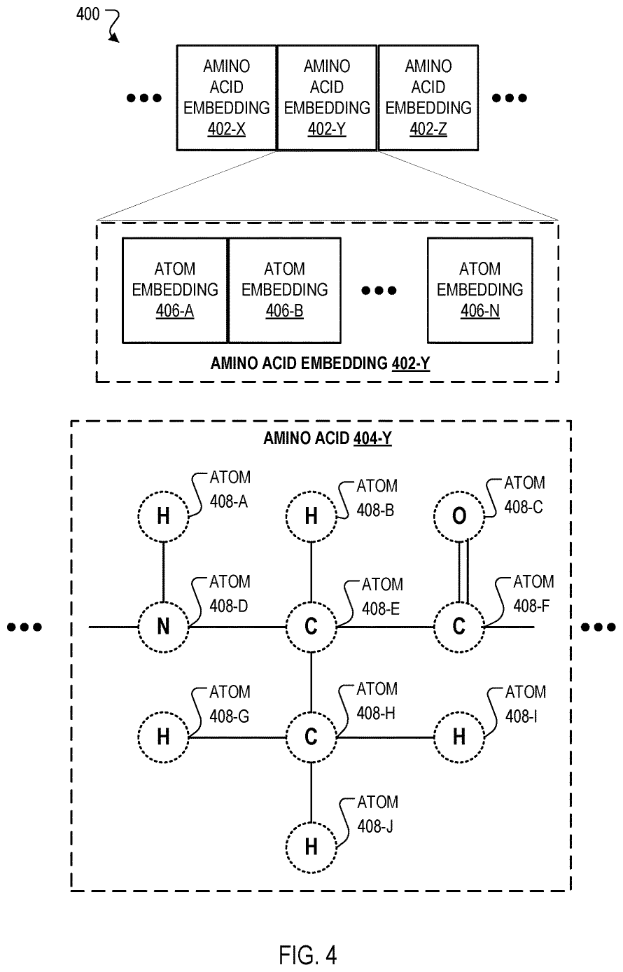

As described above, the diffusion model 104 can process noisy molecular structure data 106 to generate denoised molecular structure data 108 that characterizes a designed protein. The noisy molecular structure data 106 and the denoised molecular structure data 108 can include embeddings that characterize positions of atoms that may be included within the designed protein. Example embedding schemes for the noisy molecular structure data 106 and the denoised molecular structure data 108 are described in more detail below with reference to FIG. 4-6.

The diffusion model 104 can generate the denoised molecular structure data 108 using an iterative denoising process. In particular, the diffusion model 104 can include a denoising neural network 302 configured to denoise and update the noisy molecular structure data 106 over a sequence of denoising iterations.

At each denoising iteration, the denoising neural network 302 can process noisy molecular structure data 106 for the iteration and generate (at least partially) denoised molecular structure data 108 for the iteration. The denoising neural network 302 can use the denoised molecular structure data 108 for the iteration as an input (e.g., as the noisy molecular structure data 106) for the next denoising iteration. In some implementations, the denoising neural network 302 can generate the noisy molecular structure data 106 for the next iteration by combining random noise (e.g., as sampled from a Gaussian distribution, a uniform distribution, etc.) with the denoised molecular structure data 108 for the denoising iteration.

An example process of generating denoised molecular structure data for a protein using a denoising neural network is described in more detail below with reference to FIG. 7.

The denoising neural network 302 can have any of a variety of neural network architectures. As one example, the denoising neural network 302 can include one or more self-attention layers that can process the noisy molecular structure data 106 following a self-attention mechanism between the atom embeddings within the noisy molecular structure data 106. As another example, the denoising neural network 302 can be a graph neural network that can process an input graph representing the noisy molecular structure data 106 to generate the denoised molecular structure data 108. Other architectures for the denoising neural network 302 may also be used, such as a multi-layer perceptron architecture, a recurrent neural network architecture, and so on.

FIG. 4 illustrates an example embedding scheme that specifies the positions of atoms within amino acids for a protein.

As illustrated, the protein is represented by a sequence of amino acid embeddings 400, which includes amino acid embeddings 402-X, 402-Y, and 402-Z. Each amino acid embedding of the sequence 400 includes information that specifies a composition and spatial structure for a corresponding amino acid from the protein. For example, as illustrated, the amino acid embedding 402-Y specifies a composition and spatial structure for amino acid 404-Y of the protein. For illustrative purposes, the amino acid 404-Y is depicted as being alanine, however an amino acid embedding can characterize any of a variety of amino acids within the protein.

As illustrated, each amino acid embedding of the sequence 400 includes a plurality of atom embeddings representing a predefined set of atoms that may be included within the corresponding amino acid of the protein. For example, the amino acid embedding 402-Y includes the atom embeddings 406-A through 406-N. Each of the atom embeddings 406-A through 406-N can specify a spatial location for an atom for the amino acid 404-Y.

For example, following the embedding scheme illustrated in FIG. 4, each of the atom embeddings 406-A through 406-N can specify a spatial position of an atom that may be included within the amino acid 404-Y. The identity and spatial structure of the amino acid 404-Y can be determined by which atoms are included within the amino acid 404-Y and by the spatial positions of the included atoms represented by the atom embeddings 406-A through 406-N. As illustrated, the amino acid 404-Y includes the atoms 408-A through 408-J, whose spatial positions and elemental identities are specified by the corresponding atom embeddings 406-A through 406-J.

The predefined set of atoms for each of the amino acids of the protein can include each atom present within a predefined set of amino acids or amino acid residues. For example, the atom embeddings 406-A through 406-N can each represent an atom present within a predefined set of amino acids. As a particular example, the atom embeddings 406-A through 406-N can each represent a respective one of 37 atoms present within the following set of amino acids: alanine, cysteine, aspartic acid, glutamic acid, phenylalanine, glycine, histidine, isoleucine, lysine, leucine, methionine, asparagine, pyrrolysine, proline, glutamine, arginine, serine, threonine, selenocysteine, valine, tryptophan, and tyrosine.

In general, the predefined set of atoms can include more atoms than are present in any individual amino acid of the protein. For example, the amino acid 404-Y includes 10 of the example set of 37 atoms represented by the atom embeddings 406-A through 406-N. The system can determine which atoms are excluded from each amino acid of the protein. An example process for determining which atoms are excluded from amino acids of the protein is described in more detail below with reference to FIG. 9. As a further example, the system can use the example process of FIG. 9 to determine that the remaining 27 atoms represented by the atom embeddings 406-A through 406-N are excluded from the amino acid 404-Y.

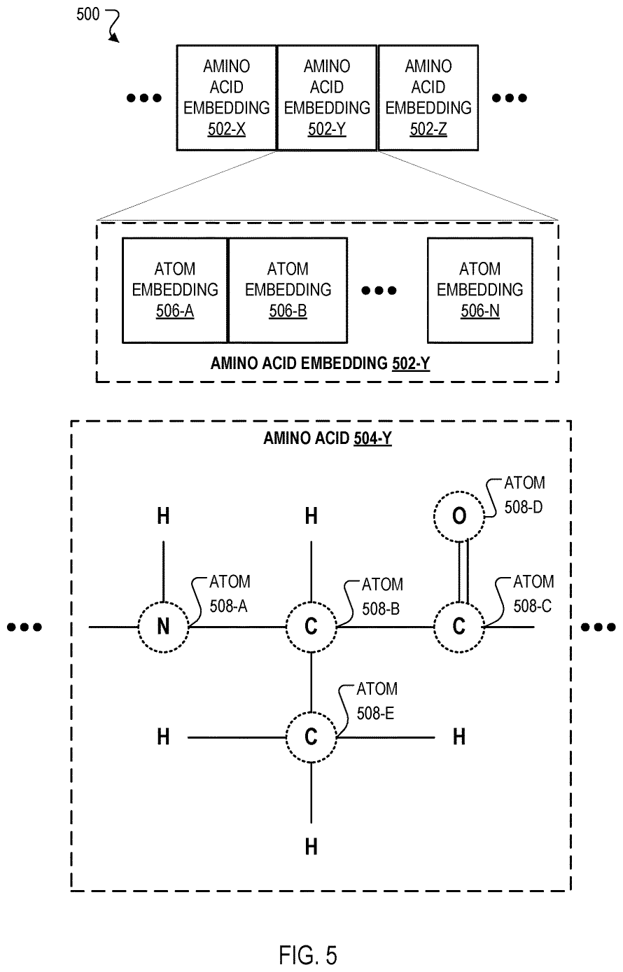

FIG. 5 illustrates an example sequence of embeddings that specify the positions of heavy atoms within amino acids for a protein.

As illustrated, the protein is represented by a sequence of amino acid embeddings 500, which includes amino acid embeddings 502-X, 502-Y, and 502-Z. Each amino acid embedding of the sequence 500 includes information that specifies a composition and spatial structure for a corresponding amino acid from the protein. For example, as illustrated, the amino acid embedding 502-Y specifies a composition and spatial structure for amino acid 504-Y of the protein. For illustrative purposes, the amino acid 504-Y is depicted as being alanine, however an amino acid embedding can characterize any of a variety of amino acids within the protein.

As illustrated, each amino acid embedding of the sequence 500 includes a plurality of atom embeddings representing a predefined set of atoms that may be included within the corresponding amino acid of the protein. For example, the amino acid embedding 502-Y includes the atom embeddings 506-A through 506-N.

In some use cases, it may not be necessary to specify positions of light atoms (e.g., hydrogen) to design the protein. For these use cases, the described systems can use an embedding scheme that specifies positions only for heavy atoms (e.g., heavier than hydrogen) within the amino acids of the protein. For example, following the embedding scheme illustrated in FIG. 5, each of the atom embeddings 506-A through 506-N can specify a spatial position of an atom that may be included within the amino acid 504-Y. The identity and spatial structure of the amino acid 504-Y can be determined by which heavy atoms are included within the amino acid 504-Y and by the spatial positions of the included atoms represented by the atom embeddings 506-A through 506-N. As illustrated, the amino acid 504-Y includes the atoms 508-A through 508-E, whose spatial positions and elemental identities are specified by the corresponding atom embeddings 506-A through 506-E.

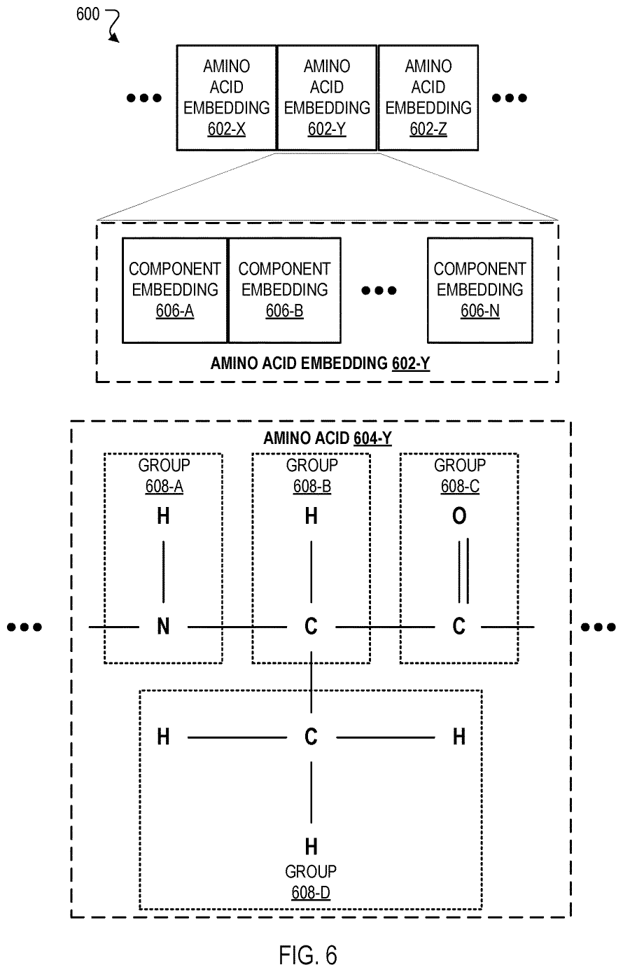

FIG. 6 illustrates an example sequence of embeddings that specify the positions of groups of atoms within amino acids for a protein.

As illustrated, the protein is represented by a sequence of amino acid embeddings 600, which includes amino acid embeddings 602-X, 602-Y, and 602-Z. Each amino acid embedding of the sequence 600 includes information that specifies a composition and spatial structure for a corresponding amino acid from the protein. For example, as illustrated, the amino acid embedding 602-Y specifies a composition and spatial structure for amino acid 604-Y of the protein. For illustrative purposes, the amino acid 604-Y is depicted as being alanine, however an amino acid embedding can characterize any of a variety of amino acids within the protein.

In some implementations, each amino acid embedding can specify the positions of amino acid components (e.g., atoms or groups of atoms) that may be included within the amino acid. As illustrated, each amino acid embedding of the sequence 600 includes a plurality of component embeddings representing a predefined set of amino acid components that may be included within the corresponding amino acid of the protein. For example, the amino acid embedding 602-Y includes the component embeddings 606-A through 606-N.

Following the embedding scheme illustrated in FIG. 6, each of the component embeddings 606-A through 606-N can specify a spatial position and a spatial orientation of a respective group of atoms (e.g., a moiety, a functional group, a side chain, etc.) that may be included within the amino acid 604-Y. The identity and spatial structure of the amino acid 604-Y can be determined by which groups of atoms are included within the amino acid 604-Y and by the spatial positions and the spatial orientations of the included groups of atoms represented by the component embeddings 606-A through 606-N. As illustrated, the amino acid 604-Y includes the groups of atoms 608-A through 608-D, whose spatial positions, spatial orientations, and elemental compositions are specified by the corresponding component embeddings 606-A through 606-D.

FIG. 7 is a flow diagram of an example process for generating molecular structure data for a protein using a denoising neural network. For convenience, the process 700 will be described as being performed by a system of one or more computers located in one or more locations. For example, a protein design system, e.g., the protein design system 100 of FIG. 1, appropriately programmed in accordance with this specification, can perform the process 700.

The system can receive noisy molecular structure data for the protein (step 702). As described above, the noisy molecular structure data can include a sequence of amino acid embeddings. Each of the amino acid embeddings can characterize a structure and composition of a corresponding amino acid within the protein.

In particular, each amino acid embedding within the noisy molecular structure data can include an atom embedding for each of a predefined set of atoms. Each atom embedding can include numerical values that characterize a spatial position for an atom that may be included within the corresponding amino acid of the protein.

In some implementations, the noisy molecular structure data can include atom embeddings that represent atoms for a target molecule. The target molecule can, for example, be a target ligand (e.g., a protein ligand or a small molecule ligand) for the protein. As another example, the target molecule can be a target protein.

In some implementations, the noisy molecular structure can designate one or more atoms as being static atoms. When the system denoises the noisy molecular structure data, the system will maintain the spatial positions for the static atoms. For example, to generate denoised molecular structure data for a protein predicted to bind with a target molecule, the noisy molecular structure data can include atom embeddings for atoms of the target molecule that are designated as static embeddings. As another example, to generate denoised molecular structure data for a complete protein that includes a particular sub-structure (e.g., a particular set of amino acid residues for the protein), the noisy molecular structure data can include atom embeddings for atoms of the particular sub-structure that are designated as static embeddings.

The system can process the noisy molecular structure data using the denoising neural network to generate denoised molecular structure data (step 704). The denoising neural network can include any of a variety of processing layers that can generate the denoised molecular structure data. For example, the denoising neural network can include feed-forward processing layers (e.g., linear layers, non-linear layers, etc.).

As another example, the denoising neural network can include one or more self-attention layers that can generate the denoised molecular structure data by applying a self-attention mechanism to the noisy molecular structure data. Each self-attention layer can update the embeddings within the noisy molecular structure data based on attention weights calculated for each pair of embeddings. As an example, a self-attention layer can calculate an updated embedding, y′, for an i-th atom embedding of the noisy molecular structure data following:

$$\vec{{y}_{ι}} = {\sum }_{j} σ ( ( Q \vec{{x}_{ι}} ) {( K \vec{{x}_{J}} )}^{T} + {b}_{i , j} ) V \vec{{x}_{J}}$$

Where Q, K, and V are learned matrices for the self-attention layer, \vec{xⱼ} is a j-th atom embedding of the input noisy molecular structure data, b_{i,j} is an attention bias between the i-th and j-th atoms, and σ is the soft-max function. In the above expression, the term (Q\vec{xᵢ}) (K\vec{xⱼ}))^{T} corresponds to an attention weight between the i-th and j-th atoms while the term σ((Q\vec{xᵢ})(K\vec{xⱼ})^{T}+b_{i,j}) corresponds to a final attention weight between the i-th and j-th atoms.

The attention bias term, b_{i,j}, can be calculated based on distances between the i-th and j-th atoms. For example, the attention bias term, b_{i,j}, can be calculated based on a spatial distance (e.g., an L2 distance) between the i-th and j-th atoms (e.g., as represented by the i-th and j-th atom embeddings within the noisy molecular structure data). As another example, the attention bias term, b_{i,j}, can be calculated based on a sequential distance between the i-th and j-th atoms (e.g., a difference between indices for the i-th and j-th atoms). As a further example, the attention bias term, b_{i,j}, can be calculated based on both a spatial and a sequential distance between the i-th and j-th atoms.

The attention bias term, b_{i,j}, may, for example, have the value of a distance, d_{i,j,} between the i-th and j-th atoms. As another example, attention bias term, b_{i,j}, may be calculated based a cutoff threshold, τ, following:

$${b}_{i , j} = \{ \begin{matrix}- {d}_{i , j} , {d}_{i , j} & \leq τ \\ - ∞ , {d}_{i , j} & > τ\end{matrix}$$

The system can determine whether denoising is complete (step 706). For example, the system can determine that denoising is complete based on a predetermined number of denoising iterations having been performed. If the system determines that denoising is not complete, the system can continue to a next denoising iteration (e.g., return to step 702). When the system continues to a next denoising iteration, the system can generate noisy molecular structure data for the next denoising iteration. As an example, the system can use the denoised molecular structure data generated during the current denoising iteration as the noisy molecular structure data for the next denoising iteration. As another example, the system can generate the noisy molecular structure data for the next denoising iteration by combining random noise (e.g., as sampled from a Gaussian distribution, from a uniform distribution, etc.) with the denoised molecular structure data generated during the current denoising iteration.

When the system determines that denoising is complete, the system can return denoised molecular structure data (step 708).

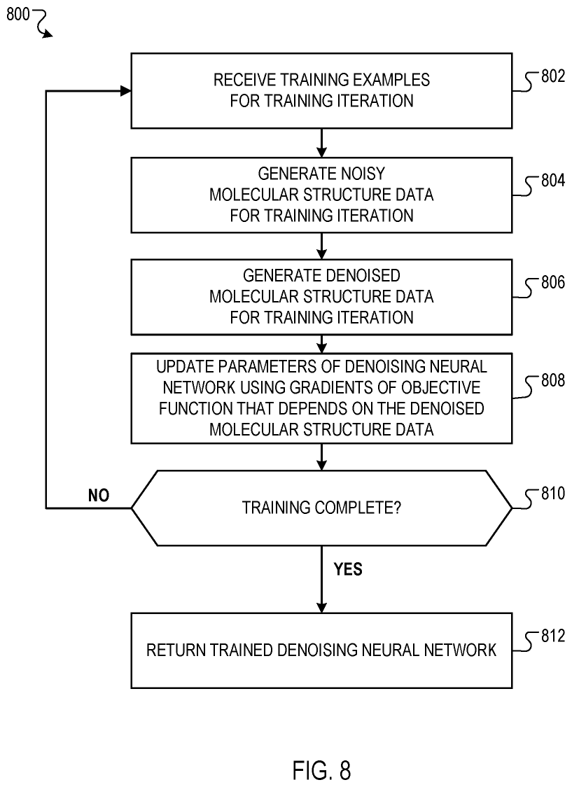

FIG. 8 is a flow diagram of an example process for training a denoising neural network to generate molecular structure data for proteins. For convenience, the process 800 will be described as being performed by a system of one or more computers located in one or more locations. For example, a protein design system, e.g., the protein design system 100 of FIG. 1, appropriately programmed in accordance with this specification, can perform the process 800.

The system can train the denoising neural network over a sequence of training iterations.

At each training iteration, the system can receive one or more training examples for the training iteration (step 802). Each training example can include data specifying target 3D spatial positions of atoms for a respective protein.

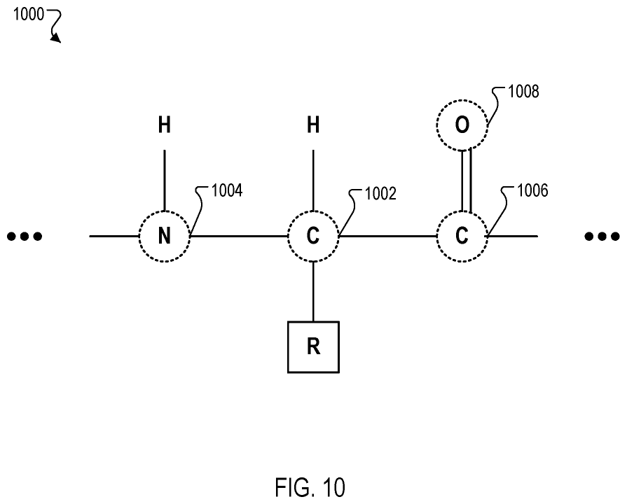

The system can generate noisy molecular structure data for the training iteration (step 804). For each training example, the system can generate a sequence of initial amino acid embeddings for the protein of the training example that characterize the target 3D spatial positions of atoms within the protein. As described above with reference to FIGS. 4-7, each amino acid embedding for a protein can include a plurality of atom embeddings representing a predefined set of atoms that may be included within the corresponding amino acid of the protein. For each amino acid of the protein for a training example, the initial amino acid embedding can be initialized to characterize: (i) the target 3D spatial positions of atoms included within the amino acid of the protein for the training example and (ii) a pre-determined throw-away location for the amino acid as the 3D spatial position of atoms that are not included within the amino acid of the protein for the training example. Examples of throw-away positions for an amino acid are described in more detail below with reference to FIG. 10.

The system generates the noisy molecular structure data for each training example by combining random noise with the initial amino acid embeddings for the training example. For instance, for each initial amino acid embedding, the system can generate a corresponding noisy amino acid embedding by adding random noise to the initial amino acid embedding.

The denoising neural network can be configured to perform a sequence of denoising iterations, e.g., as described with reference to steps 702, 704, and 706 of FIG. 7. For each training example, the system can determine a denoising iteration for the training example. As an example, for each training example, the system can randomly sample a denoising iteration for the training example from the sequence of denoising iterations, e.g., in accordance with a uniform distribution over the sequence of denoising iterations. The system can scale the random noise combined with the initial amino acid embeddings of the training example by a constant that depends on the sampled denoising iteration for the training iteration, e.g., where the values of the constants corresponding to the denoising iterations are defined by a noise schedule.

The system can then process the noisy molecular structure data using the denoising neural network to generate denoised molecular structure data for the training iteration (step 806). An example process of generating denoised molecular structure data using the denoising neural network is described in more detail above with reference to FIG. 7.

The system can determine gradients of an objective function that depends on the denoised molecular structure data and can use the gradients to update the parameter values of the denoising neural network (step 808). For each training example, the objective function can measure an error between: (i) the denoised molecular structure data generated by the denoising neural network, and (ii) a target output target output of the denoising neural network. The target output of the denoising neural network for a training example can define an output of the denoising neural network that, if used to generate an initial estimate of the 3D spatial positions of the atoms in the protein (as described in step 406 of FIG. 4), would cause the initial estimate of the 3D spatial positions of the atoms to match the target 3D spatial positions of the atoms in the protein of the training example.

The system can determine whether training is complete (step 810). If the system determines that training is not complete, the system can continue to a next training iteration (e.g., return to step 802). The system can determine whether training is complete using any of a variety of criteria. For example, the system can determine that training is complete after a pre-determined number of training iterations. As another example, the system can determine that training is complete when a value of the objective function for the training iteration falls below a pre-determined threshold. As another example, the system can determine that training is complete when a difference between the value of the objective function for the current training iteration and a value of the objective function for a previous training iteration falls below a pre-determined threshold.

When the system determines that training is complete, the system can return the trained denoising neural network (step 812).

FIG. 9 is a flow diagram of an example process for processing denoised molecular structure data to identify atoms included within amino acids for a protein. For convenience, the process 900 will be described as being performed by a system of one or more computers located in one or more locations. For example, a protein design system, e.g., the protein design system 100 of FIG. 1, appropriately programmed in accordance with this specification, can perform the process 900.

As described above, the denoised molecular structure data can include a sequence of amino acid embeddings that specify respective final spatial positions for each atom within a corresponding amino acid. In particular, each amino acid embedding can include a plurality of atom embeddings that each specify a final spatial position for a respective atom from a predefined set of atoms that may be included within the amino acid. The denoised molecular structure data can specify, based on the final spatial positions of the atoms for an amino acid of the protein, which atoms are included within the amino acid. For each amino acid within the protein, the system can process (e.g., following steps 902, 904, and 906 described below) the corresponding amino acid embedding from the denoised molecular structure data to determine which atoms are included within the amino acid.

In some implementations, the system can determine a throw-away spatial position associated with the amino acid (step 902). For example, the throw-away spatial position can be a pre-defined spatial location or region for the amino acid. Alternatively, the system may determine the throw-away spatial position based on the denoised molecular structure data. For example, the system may process the atom embeddings for the amino acid and determine the throw-away spatial position for the amino acid associated with the amino acid based on a final spatial position of a designated atom of the amino acid. Examples of throw-away spatial positions for an amino acid are described in more detail below with reference to FIG. 10.

When the system determines a throw-away spatial position for the amino acid, the system can compare the final spatial position of each atom with the throw-away spatial position (step 904). In particular, for each atom, the system can determine whether the final spatial position of the atom is within a threshold distance of the throw-away spatial position.

The system can finally determine, for each atom, whether the atom is included in the amino acid at the position in the amino acid sequence based on the final spatial position of the atom (step 906). For example, when the system determines that the final spatial position of a particular atom is within a threshold distance of the throw-away spatial position for the amino acid, the system can determine that the particular atom is not included within the amino acid.

FIG. 10 illustrates example throw-away spatial positions for an amino acid. As described above, the system can determine the throw-away spatial position for the amino acid based on final locations of a designated atom for the amino acid. In general, the designated atom for the throw-away location for the amino acid can be an atom that is present within each of a predefined set of amino acids. In particular, the designated atom for the throw-away location for the amino acid can be a backbone atom for the amino acid. For example, the designated atom can be an alpha carbon backbone atom 1002. As another example, the designated atom can be a nitrogen backbone atom 1004. As another example, the designated atom can be a carboxyl group carbon backbone atom 1006. As another example, the designated atom can be an oxygen backbone atom 1008.

FIG. 11 illustrates an example generated protein 1102 (darker in FIG. 11) designed to bind with a target protein 1104 (lighter in FIG. 11). The protein 1102 is generated by an implementation of the systems described above denoising molecular structure data specifying spatial positions for atoms of both the protein 1102 and the target protein 1104. In particular, the protein 1102 is generated by denoising spatial positions for atoms of the protein 1102 while holding positions of atoms from the target protein 1104 fixed.

FIG. 12 illustrates an example generated protein 1202 (darker in FIG. 12) designed to include a target sequence of amino acids 1204 (lighter in FIG. 12). The protein 1202 is generated by an implementation of the systems described above denoising molecular structure data specifying spatial positions for atoms of the protein 1202, including atoms of the target sequence of amino acids 1204. In particular, the protein 1202 is generated by denoising spatial positions for atoms of the protein 1202 while holding positions of atoms from the target sequence of amino acids 1204 fixed.

Some further applications of the system are described below.

The system 100 may be used to obtain a polypeptide ligand, such as polypeptide ligand that is a drug or diagnostic antibody marker of a disease, or a ligand of an industrial enzyme. For example, the noisy molecular structure data 106 can comprise data (e.g., atomic positions) defining a structure of a target molecule, which the system then uses to generate data, such as the protein design data 102, defining an amino acid sequence of the polypeptide ligand.

In some implementations the target molecule comprises a receptor or enzyme and the polypeptide ligand is an agonist or antagonist of the receptor or enzyme. In some implementations the polypeptide ligand comprises an antibody and the target molecule comprises an antibody target, in particular a virus or cancer cell protein, and the antibody binds to the antibody target to provide a therapeutic effect. In some implementations, the polypeptide ligand can be designed to bind to a cell surface marker. This may be used to identify and/or treat cancerous cells. In some implementations the target molecule may be a small molecule ligand, e.g., an organic compound with a molecular weight of <900 daltons. In some other implementations the target molecule may be a polypeptide ligand itself, i.e., defined by an amino acid sequence.

The polypeptide ligand can be synthesized and the biological activity of the polypeptide ligand can be tested in vitro and/or in vivo. For example the polypeptide ligand may be tested for ADME (absorption, distribution, metabolism, excretion) and/or toxicological properties, to screen out unsuitable ligands. The testing may comprise e.g. bringing the polypeptide ligand into contact with the target molecule and measuring a change in expression or activity of the protein. In some implementations the polypeptide ligand and/or the target molecule may comprise: an isolated antibody, a fragment of an isolated antibody, a single variable domain antibody, a bi- or multi-specific antibody, a multivalent antibody, a dual variable domain antibody, an immuno-conjugate, a fibronectin molecule, an adnectin, an DARPin, an avimer, an affibody, an anticalin, an affilin, a protein epitope mimetic or combinations thereof. The target molecule may comprise an antibody with a mutated or chemically modified amino acid Fc region, e.g. which prevents or decreases ADCC (antibody-dependent cellular cytotoxicity) activity and/or increases half-life when compared with a wild type Fc region.

The system 100 can also be used to obtain the amino acid sequence of a protein using data characterizing the structure of the protein that has been obtained by experiment, e.g., using an experimental technique that comprises one or more of: x-ray crystallography, nuclear magnetic resonance, and electron microscopy. For example, the data characterizing the structure of the protein can be provided as the conditioning data 112, which is then processed by the initialization system 110 to generate noisy molecular structure data 106 comprising spatial positions for a subset of the atoms in the protein. The denoised molecular structure data 108 may then provide a more complete or more accurate structure of the protein, which can then be processed by the structure processing system 114 to obtain data defining the amino acid sequence of the protein.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

In addition to the embodiments described above, the following embodiments are also innovative:

Embodiment 1 is a method performed by one or more computers for designing a protein, the method comprising: generating noisy molecular structure data that defines, for each position in an amino acid sequence of the protein, a corresponding initial spatial position for each atom in a predefined set of possible atoms, wherein at least some of the initial spatial positions in the noisy molecular structure data are sampled from a noise distribution; and processing the noisy molecular structure data using a diffusion model that comprises a denoising neural network to generate denoised molecular structure data that defines a denoised version of the noisy molecular structure data.

Embodiment 2 is the method of embodiment 1, further comprising processing the denoised molecular structure data to determine a respective identity of an amino acid at each of a plurality of positions in the amino acid sequence of the protein.

Embodiment 3 is the method of embodiment 1 or embodiment 2, wherein the predefined set of possible atoms includes each atom present in each amino acid in a predefined set of possible amino acids.

Embodiment 4 is the method of embodiment 3, wherein the predefined set of possible atoms includes at least 30 atoms.

Embodiment 5 is the method of embodiment 4, wherein the predefined set of possible atoms includes 37 atoms.

Embodiment 6 is the method of any one of embodiments 3-5, wherein the predefined set of possible amino acids includes at least 20 amino acids.

Embodiment 7 is the method of any one of embodiments 3-6, including the operations of the method of embodiment 2, wherein processing the denoised molecular structure data to determine the respective identity of the amino acid at each of the plurality of positions in the amino acid sequence of the protein comprises: identifying, for each of the plurality of positions in the amino acid sequence of the protein, a proper subset of the predefined set of possible atoms as being included in the amino acid at the position based on the denoised molecular structure data; and determining, for each of the plurality of positions in the amino acid sequence of the protein, the identity of the amino acid at the position based on the proper subset of the set of possible atoms that are included in the amino acid at the position.

Embodiment 8 is the method of embodiment 7, wherein for each of the plurality of positions in the amino acid sequence of the protein, determining the identity of the amino acid at the position based on the proper subset of the set of possible atoms that are included in the amino acid at the position comprises: mapping the proper subset of the set of possible atoms that are included in the amino acid at the position onto a corresponding amino acid identity in accordance with a predefined mapping.

Embodiment 9 is the method of embodiment 7 or embodiment 8, wherein the denoised molecular structure data defines, for each position in the amino acid sequence of the protein, a respective final spatial position for each atom in the predefined set of atoms; and wherein for each of the plurality of positions in the amino acid sequence of the protein, identifying a proper subset of the predefined set of possible atoms as being included in the amino acid at the position in the amino acid sequence based on the denoised molecular structure data comprises: determining, for each of a plurality of atoms in the predefined set of atoms, whether the atom is included in the amino acid at the position in the amino acid sequence based on the final spatial position of the atom.

Embodiment 10 is the method of embodiment 9, wherein determining, for each of the plurality of atoms in the predefined set of atoms, whether the atom is included in the amino acid at the position in the amino acid sequence based on the final spatial position of the atom comprises: determining a throw-away spatial position associated with the amino acid at the position in the amino acid sequence based on the denoised molecular structure data; and determining, for each of the plurality of atoms in the predefined set of atoms, whether the atom is included in the amino acid at the position based on a comparison of: (i) the final spatial position of the atom, and (ii) the throw-away spatial position associated with the amino acid at the position in the amino acid sequence.

Embodiment 11 is the method of embodiment 10, wherein the determining the throw-away spatial position associated with the amino acid at the position in the amino acid sequence based on the denoised molecular structure data comprises: determining the throw-away spatial position associated with the amino acid based on a final spatial position of a designated atom from the predefined set of possible atoms.

Embodiment 12 is the method of embodiment 11, wherein the designated atom from the predefined set of possible atoms is present in each amino acid in a predefined set of possible amino acids.

Embodiment 13 is the method of embodiment 12, wherein the designated atom from the predefined set of possible atoms is a backbone atom.

Embodiment 14 is the method of embodiment 13, wherein the designated atom from the predefined set of possible atoms is an alpha carbon backbone atom.

Embodiment 15 is the method of embodiment 13, wherein the designated atom from the predefined set of possible atoms is a nitrogen backbone atom.

Embodiment 16 is the method of embodiment 13, wherein the designated atom from the predefined set of possible atoms is an oxygen backbone atom.

Embodiment 17 is the method of any one of embodiments 10-16, wherein for one or more atoms in the predefined set of atoms, determining whether the atom is included in the amino acid at the position based on the comparison of: (i) the final spatial position of the atom, and (ii) the throw-away spatial position associated with the amino acid at the position in the amino acid sequence comprises: determining that the final spatial position of the atom is within a threshold distance of the throw-away spatial position associated with the amino acid at the position in the amino acid sequence; and in response, determining that the atom is not included in the amino acid at the position in the amino acid sequence.

Embodiment 18 is the method of any one of embodiments 1-17, wherein the noisy molecular structure data designates one or more atoms in the protein as being static atoms having predefined spatial positions that are not modified by the diffusion model.

Embodiment 19 is the method of any one of embodiments 1-18, wherein the noisy molecular structure data comprises data defining a structure of a target molecule; and wherein the protein is predicted to bind to the target molecule.

Embodiment 20 is the method of embodiment 19, wherein the target molecule is not a protein.

Embodiment 21 is the method of embodiment 19, wherein the target molecule is a protein.

Embodiment 22 is the method of any one of embodiments 19-21, wherein the noisy molecular structure data defines, for each atom in the target molecule, a corresponding initial spatial position for the atom.

Embodiment 23 is the method of any one of embodiments 19-22, wherein the noisy molecular structure data designates one or more atoms in the target molecule as being static atoms having predefined spatial positions that are not modified by the diffusion model.

Embodiment 24 is the method of any one of embodiments 19-23, wherein the noisy molecular structure data comprises a set of embeddings; wherein the set of embeddings comprises a respective embedding corresponding to each position in the amino acid sequence of the protein; wherein the set of embeddings comprises a respective embedding corresponding to each atom in the target molecule.

Embodiment 25 is the method of embodiment 24, wherein embeddings corresponding to positions in the amino acid sequence of the protein have a same dimensionality as embeddings corresponding to atoms in the target molecule.

Embodiment 26 is the method of any one of embodiments 1-25, wherein processing the noisy molecular structure data using the diffusion model to generate the denoised molecular structure data comprises iteratively denoising the noisy molecular structure data over a sequence of denoising iterations using the denoising neural network.

Embodiment 27 is the method of embodiment 26, wherein iteratively denoising the noisy molecular structure data over the sequence of denoising iterations comprises, at each denoising iteration: receiving noisy molecular structure data for the denoising iteration; and processing the noisy molecular structure data for the denoising iteration using the denoising neural network to generate denoised molecular structure data for the denoising iteration.

Embodiment 28 is the method of embodiment 27, further comprising, at each denoising iteration before a last denoising iteration in the sequence of denoising iterations: generating noisy molecular structure data for a next denoising iteration based on the denoised molecular structure data for the denoising iteration; and providing the noisy molecular structure data for the next denoising iteration.

Embodiment 29 is the method of embodiment 28, wherein at each denoising iteration before the last denoising iteration in the sequence of denoising iterations, generating the noisy molecular structure data for the next denoising iteration based on the denoised molecular structure data for the denoising iteration comprises: combining random noise with the denoised molecular structure data for the denoising iteration.

Embodiment 30 is the method of any one of embodiments 26-29, wherein at each denoising iteration: the noisy molecular structure data for the denoising iteration comprises a set of embeddings, wherein each embedding corresponds to a position in the amino acid sequence of the protein or to an atom in a target molecule; and processing the noisy molecular structure data for the denoising iteration using the denoising neural network to generate denoised molecular structure data for the denoising iteration comprises: processing the set of embeddings by one or more self-attention neural network layers of the denoising neural network.

Embodiment 31 is the method of embodiment 30, wherein processing the set of embeddings by one or more self-attention neural network layers of the denoising neural network comprises, for each self-attention neural network layer: processing the set of embeddings to generate a respective attention weight for each pair of embeddings from the set of embeddings; and generating a respective attention bias for each pair of embeddings from the set of embeddings; generating a final attention weight for each pair of embeddings from the set of embeddings based on the attention weights and the attention biases; and updating the set of embeddings using the final attention weights.

Embodiment 32 is the method of embodiment 31, wherein for each pair of embeddings from the set of embeddings, the attention bias for the pair of embeddings is based on: a spatial distance between a pair of entities represented by the pair of embeddings, or a sequence distance between a pair of entities represented by the pair of embeddings, or both.

Embodiment 33 is the method of any one of claims 3-32, including the operations of the method of embodiment 2, wherein processing the denoised molecular structure data to determine the respective identity of the amino acid at each of the plurality of positions in the amino acid sequence of the protein comprises: processing at least a portion of the denoised molecular structure using a sequencing machine learning model to generate data defining the respective identity of the amino acid at each of the plurality of positions in the amino acid sequence of the protein.

Embodiment 34 is a system comprising: one or more computers; and one or more storage devices communicatively coupled to the one or more computers, wherein the one or more storage devices store instructions that, when executed by the one or more computers, cause the one or more computers to perform operations of the respective method of any one of embodiments 1-33.

Embodiment 35 is one or more non-transitory computer storage media storing instructions that when executed by one or more computers cause the one or more computers to perform operations of the respective method of any one of embodiments 1-33.

Embodiment 36 is a method for producing a protein, comprising: generating data defining an amino acid sequence of a protein using the method of any one of embodiments 1-33; and synthesizing a protein having the amino acid sequence.

Embodiment 37 is a method of obtaining a polypeptide ligand, wherein the polypeptide ligand is a drug or diagnostic antibody marker of a disease, or a ligand of an industrial enzyme, the method comprising: generating data defining an amino acid sequence of the polypeptide ligand using the method of any one of embodiments 1-33, wherein the noisy molecular structure data comprises data defining a structure of a target molecule to which the polypeptide ligand should bind.

Embodiment 38 is the method of embodiment 37, wherein the target molecule comprises a receptor or enzyme, and wherein the polypeptide ligand is an agonist or antagonist of the receptor or enzyme; or wherein the polypeptide ligand comprises an antibody and the target molecule comprises an antibody target, in particular a virus or cancer cell protein, and wherein the antibody binds to the antibody target to provide a therapeutic effect.

Embodiment 39 is the method of embodiment 37 or 38, further comprising testing biological activity of the polypeptide ligand in vitro or in vivo.

Embodiment 40 is a method of obtaining the amino acid sequence of a protein, comprising: receiving data characterizing the structure of the protein, wherein the data has been obtained by experiment; and performing the method of any one of embodiments 1-33 to determine a predicted amino acid sequence of the protein, wherein the noisy molecular structure data comprises the data characterizing the structure of the protein.

Embodiment 41 is the method of embodiment 40, wherein the data characterizing the structure of the protein comprises spatial positions for a subset of the atoms in the protein.

Embodiment 42 is the method of embodiment 40 or embodiment 41, wherein the experimental technique comprises one or more of: x-ray crystallography, nuclear magnetic resonance, and electron microscopy.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers for designing a protein, the method comprising:
generating noisy molecular structure data that defines, for each position in an amino acid sequence of the protein, a corresponding initial spatial position for each atom in a predefined set of possible atoms,
wherein at least some of the initial spatial positions in the noisy molecular structure data are sampled from a noise distribution; and
processing the noisy molecular structure data using a diffusion model that comprises a denoising neural network to generate denoised molecular structure data that defines a denoised version of the noisy molecular structure data.

2. The method of claim 1, further comprising:
processing the denoised molecular structure data to determine a respective identity of an amino acid at each of a plurality of positions in the amino acid sequence of the protein.

3. The method of claim 1 or claim 2, wherein the predefined set of possible atoms includes each atom present in each amino acid in a predefined set of possible amino acids.

4. The method of claim 3, wherein the predefined set of possible atoms includes at least 30 atoms.

5. The method of claim 4, wherein the predefined set of possible atoms includes 37 atoms.

6. The method of any one of claims 3-5, wherein the predefined set of possible amino acids includes at least 20 amino acids.

7. The method of any preceding claim when dependent on claim 2, wherein processing the denoised molecular structure data to determine the respective identity of the amino acid at each of the plurality of positions in the amino acid sequence of the protein comprises:
identifying, for each of the plurality of positions in the amino acid sequence of the protein, a proper subset of the predefined set of possible atoms as being included in the amino acid at the position based on the denoised molecular structure data; and
determining, for each of the plurality of positions in the amino acid sequence of the protein, the identity of the amino acid at the position based on the proper subset of the set of possible atoms that are included in the amino acid at the position.

8. The method of claim 7, wherein for each of the plurality of positions in the amino acid sequence of the protein, determining the identity of the amino acid at the position based on the proper subset of the set of possible atoms that are included in the amino acid at the position comprises:
mapping the proper subset of the set of possible atoms that are included in the amino acid at the position onto a corresponding amino acid identity in accordance with a predefined mapping.

9. The method of claim 7 or claim 8, wherein the denoised molecular structure data defines, for each position in the amino acid sequence of the protein, a respective final spatial position for each atom in the predefined set of atoms; and
wherein for each of the plurality of positions in the amino acid sequence of the protein, identifying a proper subset of the predefined set of possible atoms as being included in the amino acid at the position in the amino acid sequence based on the denoised molecular structure data comprises:
determining, for each of a plurality of atoms in the predefined set of atoms, whether the atom is included in the amino acid at the position in the amino acid sequence based on the final spatial position of the atom.

10. The method of claim 9, wherein determining, for each of the plurality of atoms in the predefined set of atoms, whether the atom is included in the amino acid at the position in the amino acid sequence based on the final spatial position of the atom comprises:
determining a throw-away spatial position associated with the amino acid at the position in the amino acid sequence based on the denoised molecular structure data; and
determining, for each of the plurality of atoms in the predefined set of atoms, whether the atom is included in the amino acid at the position based on a comparison of: (i) the final spatial position of the atom, and (ii) the throw-away spatial position associated with the amino acid at the position in the amino acid sequence.

11. The method of claim 10, wherein the determining the throw-away spatial position associated with the amino acid at the position in the amino acid sequence based on the denoised molecular structure data comprises:
determining the throw-away spatial position associated with the amino acid based on a final spatial position of a designated atom from the predefined set of possible atoms.

12. The method of claim 11, wherein the designated atom from the predefined set of possible atoms is present in each amino acid in a predefined set of possible amino acids.

13. The method of claim 12, wherein the designated atom from the predefined set of possible atoms is a backbone atom.

14. The method of claim 13, wherein the designated atom from the predefined set of possible atoms is an alpha carbon backbone atom.

15. The method of claim 13, wherein the designated atom from the predefined set of possible atoms is a nitrogen backbone atom.

16. The method of claim 13, wherein the designated atom from the predefined set of possible atoms is an oxygen backbone atom.

17. The method of any one of claims 10-16, wherein for one or more atoms in the predefined set of atoms, determining whether the atom is included in the amino acid at the position based on the comparison of: (i) the final spatial position of the atom, and (ii) the throw-away spatial position associated with the amino acid at the position in the amino acid sequence comprises:
determining that the final spatial position of the atom is within a threshold distance of the throw-away spatial position associated with the amino acid at the position in the amino acid sequence; and
in response, determining that the atom is not included in the amino acid at the position in the amino acid sequence.

18. The method of any preceding claim, wherein the noisy molecular structure data designates one or more atoms in the protein as being static atoms having predefined spatial positions that are not modified by the diffusion model.

19. The method of any preceding claim, wherein the noisy molecular structure data comprises data defining a structure of a target molecule; and
wherein the protein is predicted to bind to the target molecule.

20. The method of claim 19, wherein the target molecule is not a protein.

21. The method of claim 19, wherein the target molecule is a protein.

22. The method of any one of claims 19-21, wherein the noisy molecular structure data defines, for each atom in the target molecule, a corresponding initial spatial position for the atom.

23. The method of any one of claims 19-22, wherein the noisy molecular structure data designates one or more atoms in the target molecule as being static atoms having predefined spatial positions that are not modified by the diffusion model.

24. The method of any one of claims 19-23, wherein the noisy molecular structure data comprises a set of embeddings;
wherein the set of embeddings comprises a respective embedding corresponding to each position in the amino acid sequence of the protein;
wherein the set of embeddings comprises a respective embedding corresponding to each atom in the target molecule.

25. The method of claim 24, wherein embeddings corresponding to positions in the amino acid sequence of the protein have a same dimensionality as embeddings corresponding to atoms in the target molecule.

26. The method of any preceding claim, wherein processing the noisy molecular structure data using the diffusion model to generate the denoised molecular structure data comprises iteratively denoising the noisy molecular structure data over a sequence of denoising iterations using the denoising neural network.

27. The method of claim 26, wherein iteratively denoising the noisy molecular structure data over the sequence of denoising iterations comprises, at each denoising iteration:
receiving noisy molecular structure data for the denoising iteration; and
processing the noisy molecular structure data for the denoising iteration using the denoising neural network to generate denoised molecular structure data for the denoising iteration.

28. The method of claim 27, further comprising, at each denoising iteration before a last denoising iteration in the sequence of denoising iterations:
generating noisy molecular structure data for a next denoising iteration based on the denoised molecular structure data for the denoising iteration; and
providing the noisy molecular structure data for the next denoising iteration.

29. The method of claim 28, wherein at each denoising iteration before the last denoising iteration in the sequence of denoising iterations, generating the noisy molecular structure data for the next denoising iteration based on the denoised molecular structure data for the denoising iteration comprises:
combining random noise with the denoised molecular structure data for the denoising iteration.

30. The method of any one of claims 26-29, wherein at each denoising iteration:
the noisy molecular structure data for the denoising iteration comprises a set of embeddings, wherein each embedding corresponds to a position in the amino acid sequence of the protein or to an atom in a target molecule; and
processing the noisy molecular structure data for the denoising iteration using the denoising neural network to generate denoised molecular structure data for the denoising iteration comprises:
processing the set of embeddings by one or more self-attention neural network layers of the denoising neural network.

31. The method of claim 30, wherein processing the set of embeddings by one or more self-attention neural network layers of the denoising neural network comprises, for each self-attention neural network layer:
processing the set of embeddings to generate a respective attention weight for each pair of embeddings from the set of embeddings; and
generating a respective attention bias for each pair of embeddings from the set of embeddings;
generating a final attention weight for each pair of embeddings from the set of embeddings based on the attention weights and the attention biases; and
updating the set of embeddings using the final attention weights.

32. The method of claim 31, wherein for each pair of embeddings from the set of embeddings, the attention bias for the pair of embeddings is based on: a spatial distance between a pair of entities represented by the pair of embeddings, or a sequence distance between a pair of entities represented by the pair of embeddings, or both.

33. The method of any preceding claim when dependent on claim 2, wherein processing the denoised molecular structure data to determine the respective identity of the amino acid at each of the plurality of positions in the amino acid sequence of the protein comprises:
processing at least a portion of the denoised molecular structure using a sequencing machine learning model to generate data defining the respective identity of the amino acid at each of the plurality of positions in the amino acid sequence of the protein.

34. A system comprising:
one or more computers; and
one or more storage devices communicatively coupled to the one or more computers, wherein the one or more storage devices store instructions that, when executed by the one or more computers, cause the one or more computers to perform operations of the respective method of any one of claims 1-33.

35. One or more non-transitory computer storage media storing instructions that when executed by one or more computers cause the one or more computers to perform operations of the respective method of any one of claims 1-33.

36. A method for producing a protein, comprising:
generating data defining an amino acid sequence of a protein using the method of any one of claims 1-33; and
synthesizing a protein having the amino acid sequence.

37. A method of obtaining a polypeptide ligand, wherein the polypeptide ligand is a drug or diagnostic antibody marker of a disease, or a ligand of an industrial enzyme, the method comprising:
generating data defining an amino acid sequence of the polypeptide ligand using the method of any one of claims 1-33, wherein the noisy molecular structure data comprises data defining a structure of a target molecule to which the polypeptide ligand should bind.

38. A method as claimed in claim 37 wherein the target molecule comprises a receptor or enzyme, and wherein the polypeptide ligand is an agonist or antagonist of the receptor or enzyme; or wherein the polypeptide ligand comprises an antibody and the target molecule comprises an antibody target, in particular a virus or cancer cell protein, and wherein the antibody binds to the antibody target to provide a therapeutic effect.

39. A method of claim 37 or 38, further comprising testing biological activity of the polypeptide ligand in vitro or in vivo.

40. A method of obtaining the amino acid sequence of a protein, comprising:
receiving data characterizing the structure of the protein, wherein the data has been obtained by experiment; and
performing the method of any one of claims 1-33 to determine a predicted amino acid sequence of the protein, wherein the noisy molecular structure data comprises the data characterizing the structure of the protein.

41. The method of claim 40, wherein the data characterizing the structure of the protein comprises spatial positions for a subset of the atoms in the protein.

42. The method of claim 40 or 41, wherein the experimental technique comprises one or more of: x-ray crystallography, nuclear magnetic resonance, and electron microscopy.
